(19) United States
(12) Patent Application Publication
(10) Pub. No.: US 2005/0038410 A1
(43) Pub. Date: Feb. 17, 2005

(54) Rotatable infusion sheath apparatus

(76) Inventors: Paul A. Friedman, Rochester, MN (US); Charles J. Bruce, Rochester, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/830,816

(22) Filed: Apr. 23, 2004

Related U.S. Application Data
(60) Provisional application No. 60/464,835, filed on Apr. 23, 2003.

(51) Int. Cl. A61M 25/00

(52) U.S. Cl. 604/523

(57) ABSTRACT

The invention provides a rotatable infusion sheath apparatus. An apparatus of the invention includes a hub comprising a hub body with a longitudinal bore, at least one infusion inlet in fluid communication with the bore, and at least one elongate catheter sheath attached to the hub body. Generally, the sheath is rotatable with respect to the hub body and forms a substantially fluid- and gas-impervious seal therewith. Articles of manufacture are provided that include a rotatable infusion sheath apparatus of the invention and a catheter.

### CROSS REFERENCE TO RELATED APPLICATIONS

This application claims priority under 35 U.S.C. §119(e) of U.S. Application No. 60/464,835, filed Apr. 23, 2003.

### TECHNICAL FIELD

This invention relates to sheaths, and more particularly to a rotatable infusion sheath apparatus.

### BACKGROUND

Interventional catheter-based procedures have lead to dramatic advances in the care of a number of diseases. Such procedures include diagnostic angiography and angioplasty and stenting for coronary artery disease, carotid disease, and peripheral vascular disease; balloon valvuloplasty for mitral stenosis and aortic stenosis; closure devices for repair of atrial and ventricular septal defects; intravascular coil placement for arteriovenous fistulae; local therapy delivery of many different modalities for treatment of malignancy; and radiofrequency catheter ablation for arrhythmias.

While these procedures have offered significant advances in the delivery of medical care, they share common limitations. Percutaneous catheter interventions, particularly within the vascular system, have been complicated by thromboembolism and air embolism, which may be associated with stroke or myocardial infarction. Moreover, the recent successful use of catheter ablation for the elimination of atrial fibrillation has led to increased left atrial procedures. Atrial procedures may at times be prolonged, with a potentially greater stroke and air embolism risk. In addition, the need for contrast agents for imaging in most procedures (for example, venography in pulmonary vein isolation procedures) may result in inadvertent entry of air into the long sheaths used to access the left atrium, leading to air embolism, or dislodgment of thrombus.

Currently, all commercially available long sheaths have a side port to permit infusion of a solution, generally for flushing or irrigation purposes. However, continuous infusion of a solution (e.g., a saline solution) or of a contrast agent through the side port limits sheath mobility, and likewise, catheter mobility. Rotational movement of long sheaths is often required to properly position the catheter for ablation and for angiography or venography. However, when the sheath is attached to side infusion tubing that may be connected to, for example, pressure bags outside of the sterile field, sheath rotation leads to knotting and entanglement of the attached tubing. Furthermore, if two or more sheaths require irrigation (as is typically the case during atrial fibrillation ablation), there is greater mechanical complexity and entanglement of the infustion tubing.

A device that facilitates intravascular and intracavitary access while minimizing 10 both the entanglement of infusion tubing and the risk of thrombus formation, thomboembolism, or air embolism is desirable. The rotatable infusion sheath apparatus described herein is such a device. The apparatus described herein is useful in many body cavities and spaces, and across many fields of medicine.

### SUMMARY

An apparatus of the invention includes a sheath and a hub. A rotatable connection between the sheath and the hub allows for rotation and manipulation of the sheath and catheter while the hub remains stationary. The configuration of the hub and sheath that allows for independent rotation of the sheath means that infusion tubing that is connected to an infusion inlet on the hub does not get tangled during catheterization or other procedures. The configuration of the hub and the sheath further provides for a rotatable seal that is airtight and liquidtight. Such a seal, in combination with positive pressure. infusion, reduces the possibility of air bubbles entering the sheath, which can be potentially lethal to a patient.

In one aspect, the invention provides a rotatable infusion sheath apparatus that includes a hub. The hub generally comprises a hub body with a longitudinal bore; at least one infusion inlet in fluid communication with the bore; and at least one elongate catheter sheath attached to the hub body. Typically, the bore has a first end forming a catheter inlet port and a second end forming a catheter outlet port. Generally, the elongate catheter sheath is attached to the hub body at the catheter outlet port. It is a feature of the invention that the sheath is rotatable with respect to the hub body around a longitudinal axis of the bore, and additionally, that the sheath form a substantially fluid and gas impervious seal therewith. The apparatus can further include a substantially liquid and gas impervious diaphragm in the bore of the hub body proximal the catheter inlet port.

In some embodiments, the hub body comprises a stationary seat proximal the catheter outlet port. The stationary seat is generally circumferential with respect to the bore in the hub body and substantially normal to the longitudinal axis of the bore. The sheath typically comprises a substantially tubular body having a first end with a rotatable sealing member. The rotatable sealing member is generally circumferential with respect to a longitudinal axis of the sheath body and substantially normal to a longitudinal axis of the sheath. In this embodiment, the rotatable sealing member engages the stationary seat in the hub body.

In some embodiments, the hub body further comprises a sheath guide flange between the stationary seat and the catheter outlet port. The sheath guide flange is generally circumferential with respect to the bore in the hub body and substantially normal to the longitudinal axis of the bore. The rotatable sealing member can further include a hub guide flange that engages the sheath guide flange in the hub body. Typically, the hub guide flange is circumferential with respect to a longitudinal axis of the sheath body and substantially parallel to the rotatable sealing member. In certain embodiments, the hub guide flange is integral with the rotatable sealing member.

In some embodiments, the sheath further comprises a grip on an exterior surface thereof. In certain other embodiments, the tubular body and/or the sheath can have multiple lumens.

In another aspect of the invention, there is provided an article of manufacture. Articles of manufacture include a rotatable infusion sheath apparatus and a catheter. Articles of manufacture of the invention can further include an infusion injector. An infusion injector included with an article of manufacture of the invention can contain heparinized saline and/or a contrast agent.

In yet another aspect of the invention, there is provided a method of delivering therapy to a target site. Such a method includes introducing a rotatable infusion sheath apparatus into the vasculature or a body cavity of an individual; inserting a catheter into the rotatable infusion sheath apparatus and positioning the catheter near a target site; and delivering therapy to the target site. It is a feature of the invention that the hub body is stationary with respect to both the sheath and the catheter during positioning of the catheter and during the delivery of therapy. The method can further include establishing positive pressure in the apparatus by infusing fluids into the apparatus through the infusion inlet. Positive pressure is usually established prior to introducing the apparatus into an individual.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a cross-sectional image of the proximal end of a rotatable infusion sheath apparatus, wherein the sheath is shown rotated 35° with respect to the hub, while the hub remains stationary.

FIG. 2 is the rotatable infusion sheath apparatus of FIG. 1 into which a catheter has been introduced, and shows that the sheath and catheter can still be rotated via the grip, thereby facilitating catheter positioning at the target site.

Like reference symbols in the various drawings indicate like elements.

### DETAILED DESCRIPTION

An apparatus of the invention includes a sheath rotatably connected to a hub, such that during a procedure in which a sheath is used to introduce a catheter into a body, the sheath and catheter can be manipulated and rotated as needed while the hub remains stationary. The hub described herein includes an infusion inlet to receive infusion fluids, usually through infusion tubing. The configuration of the apparatus that allows for independent rotation of the sheath relative to the hub means that the infusion tubing does not get tangled during catheterization or other procedures. The configuration of the hub and the sheath further provides for a rotatable seal that is airtight and liquidtight. Such a seal, in combination with positive pressure infusion, reduces or eliminates the possibility of air bubbles entering the sheath, which can result in air embolism in the patient. Positive pressure infusion, for example, of heparinized saline, also reduces the risk of thrombus formation distally and permits the use of catheters that are smaller than the sheath (for example, to facilitate injection of contrast agents for imaging purposes) while preventing thrombus formation. The apparatus of the invention, therefore, provides several advantages over the current sheaths.

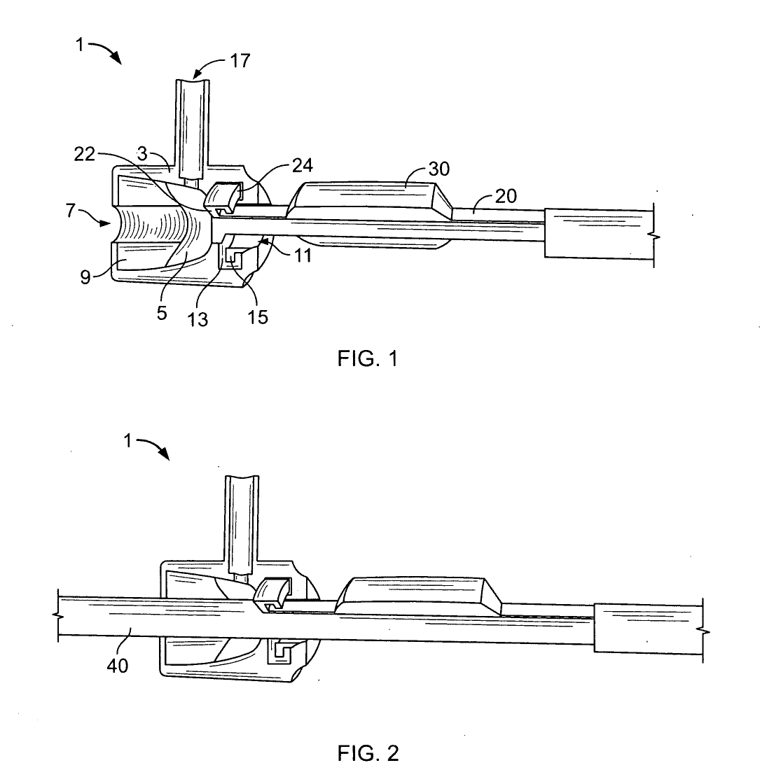

FIG. 1 shows a rotatable infusion sheath apparatus 1 of the invention, while FIG. 2 shows a catheter 40 inserted into the apparatus 1 of FIG. 1. A rotatable infusion sheath apparatus 1 as shown in FIGS. 1 and 2 includes a hub 3 and a sheath 20. A hub body 3 generally has a single longitudinal bore 5. In some embodiments, a hub body 3 can have more than one longitudinal bore for introducing at least one catheter and, for example, one or more devices for imaging and/or monitoring (e.g., angioplasty wires, or angioplasty balloons). On one end, the hub 3 has a catheter inlet port 7 for inserting a catheter into the hub 1. Proximal to the catheter inlet port 7 is a substantially liquid- and gas-impervious diaphragm 9. The diaphragm 9 at the catheter inlet port 7 permits catheter insertion (and removal) without permitting entry of air into the apparatus and into the patient.

The hub also has a catheter outlet port 11. A catheter exits the hub and enters the sheath at the catheter outlet port 11. Proximal to the catheter outlet port 11 on the hub body 3 is a stationary seat 13. A stationary seat 13 is circumferential with respect to the bore 5 and substantially normal to the longitudinal axis of the bore 5. A hub body 3 further can include a sheath guide flange 15 positioned between the stationary seat 13 and the catheter outlet port 11. A sheath guide flange 15 is generally circumferential with respect to the bore 5 and substantially normal to the longitudinal axis of the bore 5. A rotatable infusion sheath apparatus 1 of the invention also includes an infusion inlet 17 for the introduction of fluids, such as saline solutions, and contrast agents into the sheath body 20. An infusion inlet 17 as described herein is in fluid communication with the bore 5. The infusion inlet 17 is shown in FIG. 1 as an integral component of the hub body 3. Other suitable locations for the infusion inlet 17 on the hub would be apparent to those of ordinary skill in the art provided that the infusion inlet 17 is in fluid communication with the bore 5. It is intended that the infusion inlet 17 allow for entry of fluids into the sheath body 20.

A rotatable infusion sheath apparatus 1 of the invention also includes at least one elongate catheter sheath 20 attached to the hub body 3 at the catheter outlet port 11. A sheath used in an apparatus of the invention generally has a tubular body. In one embodiment, an apparatus of the invention includes one sheath 20 (as shown in FIGS. 1 and 2). A sheath suitable for use in the invention can have a single internal lumen (as shown in FIGS. 1 and 2) or multiple lumens. A sheath containing multiple lumens can accommodate multiple catheters and/or one or more devices for imaging or monitoring (e.g., an intracardiac echo device (ICE)). In other embodiments, an apparatus of the invention can include multiple sheaths attached to a single hub, or multiple sheaths attached to multiple hubs. In an embodiment wherein multiple sheaths are attached to a single hub, the hub can have multiple longitudinal bores, wherein each sheath is associated with a different longitudinal bore. The invention also provides for sheaths having various curvatures, lengths, and diameters. It is to be understood that an apparatus of the invention is not to be limited to a particular configuration of a sheath, as essentially any sheath can be adapted to be rotatably connected to a hub.

As described herein, a sheath and a hub are joined by a unique connection that permits the sheath to rotate while the hub remains stationary. It is a feature of the invention that the sheath is able to rotate with respect to the hub around a longitudinal axis of the bore. The ability of a sheath to rotate independent of a hub allows for mobility of the intracardiac portion of a sheath without twisting or entanglement of infusion tubing attached to a hub. It is an additional feature of the invention that the sheath and the hub form a substantially fluid- and gas-impervious seal therebetween. Thus, the apparatus is designed to permit constant fluid infusion without introducing any air. It is understood that connections between a sheath and a hub other than those described herein can be designed for use in an apparatus of the invention provided that the connection allows for rotation of the sheath independent of the hub while providing an airtight and liquidtight seal between the sheath and the hub.

A sheath for use in an apparatus of the invention generally has a rotatable sealing member 22 on the end that is exterior to the patient's body. A rotatable sealing member 22 is generally circumferential with respect to the longitudinal axis of the sheath body 20 and is substantially normal to the longitudinal axis of the sheath body 20. In the embodiment shown in FIGS. 1 and 2, the rotatable sealing member 22 engages the stationary seat 13 in the hub body 3. A rotatable sealing member 22 can additionally include a hub guide flange 24 that engages the sheath guide flange 15 on the hub body 3. A hub guide flange 24 generally is circumferential with respect to the longitudinal axis of the sheath body 20 and is substantially parallel to the rotatable sealing member 22. In some embodiments, the hub guide flange 24 is integral with the rotatable sealing member 22.

A rotatable infusion sheath apparatus 1 of the invention also can include a grip 30 on the exterior of the sheath 20 for manipulating the sheath 20 and/or a catheter 40 within the sheath 20. The grip and the hub described herein provide reinforcement of the proximal components of the sheath to thereby facilitate rotational motion and to provide greater rotational torque than existing sheaths, which often become twisted or kinked with excessive rotational motion. Furthermore, other embodiments, including steerability of the sheath by means of pull-wires, or the use of smaller sheaths within the apparatus to facilitate more complex three-dimensional maneuvering, also are provided by the invention.

In addition, the invention provides for articles of manufacture. An article of manufacture of the invention can include a rotatable infusion sheath apparatus 1 as described above, and a catheter. Such articles of manufacture can include any type of catheter such as those used in electrophysiological applications, interventional cardiology applications, invasive radiology, and other percutaneous invasive disciplines. St. Jude Medical (St. Paul, Minn.) supplies electrophysiology catheters as well as cardiology and vascular access catheters that can be used with a rotatable infusion sheath apparatus described herein. Such catheters include, for example, eValuator™ Electrophysiology Catheters, Livewire™ Steerable Catheters, Livewire TC™ Ablation Catheters, Supreme™ Catheters, Response™ Catheters, Pacel™ Bipolar Pacing Catheters, and Spyglass™ Angiography Catheters. In addition, electrophysiology and other catheters that can be used in a rotatable infusion sheath apparatus of the invention are commercially available from, for example, C. R. Bard, Inc. (Murray Hill, N.J.), Medtronic (Minneapolis, Minn), Cordis Corp. (Miami, Fla.), or Biosense Webster (Diamond Bar, Calif.).

Articles of manufacture containing a rotatable infusion sheath apparatus 1 as described herein can further include one or more infusion injectors. Infusion injectors for use with a rotatable infusion sheath apparatus 1 of the invention can be, for example, pre-packaged syringes or pressure bags that contain an infusion solution. According to the invention, infusion solutions can be saline solutions (e.g., normal saline, or heparinized saline), glycoprotein 2a/3b inhibitors, or a contrast agent or dye. Such pre-packaged syringes or pressure bags can be connected to the infusion inlet 17 using, for example, a syringe-infusion inlet adapter, or tubing that connects the syringe or pressure bag to the infusion inlet 17.

An apparatus of the invention can be used in many types of medical procedures, including, but not limited to, pulmonary vein isolation ablation, catheter ablation for the treatment of accessory pathways or atrial tachycardias, coronary sinus venography for placement of permanent pacing leads and biventricular pacing, and interventional procedures including mitral valvuloplasty, aortic valvuloplasty, and angioplasty of coronary arteries. Additionally, an apparatus of the invention can be used in procedures such as multiple interventional radiology procedures including aortic angiography, renal angiography and dilatation, CNS and hepatobiliary diagnostic and interventional procedures, peripheral vascular diagnostic and interventional procedures. A rotatable infusion sheath apparatus of the invention can be configured to deliver a stent, for example. An apparatus of the invention also can be configured to be used in a large variety of diagnostic and therapeutic procedures.

A method for using a rotatable infusion sheath apparatus 1 of the invention generally includes establishing positive pressure in the rotatable infusion sheath apparatus by infusing fluids into the apparatus through the infusion inlet; introducing and advancing the distal end of the apparatus 1 into the vasculature or body cavity of an individual at a position near the target site; inserting a catheter 40 into the apparatus 1 and positioning the catheter 40 at the target site; and delivering therapy to the target site. It is a feature of the invention that the hub body 3 is stationary with respect to the sheath body 20 and the catheter 40 while the catheter is being positioned and while the therapy is being delivered.

Inserting and advancing a catheter or other device into the vasculature are well-known and routine techniques used in the art. A catheter or other device is generally introduced and advanced into the vasculature of an individual using one or more guiding sheaths. Sheath designs for use in ablation procedures in both the right and/or left atrial chambers are disclosed in U.S. Pat. Nos. 5,427,119; 5,497,119; 5,564,440; and 5,575,766. The “Seldinger” technique is routinely used for introducing a sheath into the vasculature of an individual such that a catheter or other device can be advanced into the right venous system. Advancing a catheter or other device into the left atrium from the right vasculature requires traversing the septal wall. A transseptal puncture is generally performed using a “Brochenbrough” needle or trocar in an art-known procedure. It is contemplated, however, that other methods for introducing a catheter or other device via the rotatable infusion sheath apparatus of the invention into the left atrium are suitable and include, for example, a retrograde approach or a venous cut-down approach. See, for example, U.S. Pat. No. 6,254,599 for a detailed description of procedures used in the art to access the left atrium.

Following appropriate positioning, for example, of an ablation catheter, ablation elements on the catheter are energized to a sufficient level to ablate the contacted tissue. In an embodiment in which radiofrequency signals are used to generate heat at the site of ablation, a pulmonary vein ostium can be ablated for 30-120 seconds or longer at a temperature of about 40° C. to about 70° C.

An apparatus of the invention can be used to deliver a catheter via the vasculature to, without limitation, the heart, the brain, the liver, and the kidneys. In addition, introducing and advancing a catheter or other device into a non-vasculature body cavity are known techniques in the art. Body cavities into which an apparatus of the invention could be used to introduce a catheter or other device include, but are not limited to, the uterus (e.g., for treatment of endometriosis), or the gastrointestinal tract, for example.

The use of constant positive-pressure fluid infusion during a procedure (e.g., sheath introduction, treatment, and sheath withdrawal) ensures that no air enters the tubing, thereby limiting the risk of embolization. Furthermore, constant infusion of heparinized saline along the sheath body and out the distal end of the sheath limits the risk of thrombus formation, particularly at the susceptible sheath-catheter interface. It is desirable that infusion maintains positive pressure at all times. With a stationary catheter, the infusion rate can be slow (e.g., 3-5 cc/hour), but the infusion rate can increase to maintain positive pressure when, for example, a catheter is withdrawn from the sheath. Catheter withdrawal in current systems is associated with creation of a negative pressure within the sheath, leading to entrapment of air within the sheath. This problem is eliminated in the present apparatus.

In addition to or in place of a catheter, one or more devices for imaging or monitoring can be introduced using a rotatable infusion sheath apparatus 1 of the invention. Signals within the pulmonary vein can be monitored to localize an arrhythmogenic origin of the atrial arrhythmia and determine the best location to ablate and produce a conduction block. In addition, signals from within the pulmonary vein can be monitored during ablation. Ultrasound imaging of the pulmonary vein, for example, can be performed via an ICE device. The rotatable infusion sheath apparatus 1 also can be configured to perform pacing to determine whether conduction is present or whether the vein has been electrically isolated.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A rotatable infusion sheath apparatus, comprising:
a hub comprising a hub body with a longitudinal bore, wherein the bore has a first end forming a catheter inlet port and a second end forming a catheter outlet port;
at least one infusion inlet in fluid communication with the bore; and
at least one elongate catheter sheath attached to the hub body at the catheter outlet port,
wherein the sheath is rotatable with respect to the hub body around a longitudinal axis of the bore and forms a substantially fluid and gas impervious seal therewith.

2. The apparatus of claim 1, wherein the hub body comprises a stationary seat proximal the catheter outlet port, wherein the seat is circumferential with respect to the bore in the hub body and substantially normal to the longitudinal axis of the bore.

3. The apparatus of claim 2, wherein the sheath comprises a substantially tubular body having a first end with a rotatable sealing member, wherein the rotatable sealing member is circumferential with respect to a longitudinal axis of the sheath body and substantially normal to a longitudinal axis of the sheath, and wherein the rotatable sealing member engages the stationary seat in the hub body.

4. The apparatus of claim 3, wherein said tubular body comprises multiple lumens.

5. The apparatus of claim 3, wherein said sheath comprises multiple lumens.

6. The apparatus of claim 3, wherein the hub body further comprises a sheath guide flange between the stationary seat and the catheter outlet port, wherein the sheath guide flange is circumferential with respect to the bore in the hub body and substantially normal to the longitudinal axis of the bore.

7. The apparatus of claim 6, wherein the rotatable sealing member further comprises a hub guide flange that engages the sheath guide flange in the hub body.

8. The apparatus of claim 7, wherein the hub guide flange is circumferential with respect to a longitudinal axis of the sheath body and substantially parallel to the rotatable sealing member.

9. The apparatus of claim 8, wherein the hub guide flange is integral with the rotatable sealing member.

10. The apparatus of claim 1, further comprising a substantially liquid and gas impervious diaphragm in the bore of the hub body and proximal the catheter inlet port.

11. The apparatus of claim 1, wherein the sheath further comprises a grip on an exterior surface thereof.

12. An article of manufacture, comprising:
the rotatable infusion sheath apparatus of claim 1; and
a catheter.

13. The article of manufacture of claim 12, further comprising:
an infusion injector.

14. The article of manufacture of claim 13, wherein said infusion injector comprises heparinized saline and/or a contrast agent.

15. A method of delivering therapy to a target site, said method comprising:
introducing the rotatable infusion sheath apparatus of claim 1 into vasculature or a body cavity of an individual;
inserting a catheter into said rotatable infusion sheath apparatus and positioning said catheter near a target site; and
delivering therapy to said target site;
wherein said hub body is stationary with respect to said sheath and said catheter during said positioning of said catheter and during said delivery of therapy.

16. The method of claim 15, further comprising:
establishing positive pressure in said apparatus by infusing fluids into said apparatus through said infusion inlet, wherein said positive pressure is established prior to said introducing step.
